(19) United States
(12) Patent Application Publication
(10) Pub. No.: US 2022/0341863 A1
(43) Pub. Date: Oct. 27, 2022

(54) GAS DETECTION SYSTEM AND GAS DETECTION METHOD

(71) Applicant: Hitachi, Ltd., Tokyo (JP)

(72) Inventors: Hironori WAKANA, Tokyo (JP); Masuyoshi YAMADA, Tokyo (JP)

(21) Appl. No.: 17/705,532

(22) Filed: Mar. 28, 2022

(30) Foreign Application Priority Data
Apr. 27, 2021 (JP) 2021-075204

(51) Int. Cl. G01N 27/12

(52) U.S. Cl. CPC G01N 27/12

(57) ABSTRACT

An object of the invention is to provide a gas detection system that does not require customization of an odor sensor depending on an environment. The gas detection system includes a sensor array including a plurality of sensors having different characteristic sensitivities, a pre-processing unit configured to convert each of sensor output values acquired from the sensors constituting the sensor array into an output ratio as a ratio of the output value to a predetermined value, and an odor determination processing unit configured to determine, by comparing a normal range generated based on a result of executing cluster analysis on an output ratio acquired from each of the sensors constituting the sensor array in the past with the output ratio obtained by the pre-processing unit executing the conversion, whether the output ratio is within the normal range.

### CROSS-REFERENCE TO RELATED APPLICATION

The present application claims priority from Japanese application JP2021-075204, filed on Apr. 27, 2021, the contents of which is hereby incorporated by reference into this application.

### BACKGROUND OF THE INVENTION

### 1. Field of the Invention

The present invention relates to a gas detection system and a gas detection method.

### 2. Description of the Related Art

In addition to a device such as an on-site fire alarm, technical development of an odor measurement device using a combination of a plurality of odor sensors having different sensitivities has progressed. As such a technique, a method for identifying an odor type based on output patterns of a plurality of odor sensors and a method for executing machine learning on sensor data and analyzing a component have been proposed.

As such a technique, JP-A-2018-194314 discloses that “a gas analysis device 100 includes: a chamber 10; three or more gas sensors 30a, 30b, and 30c that are provided in the chamber and that include gas sensitive members having different material compositions; a storage unit 63 that stores in advance information for converting responses of the gas sensitive members of the three or more gas sensors into concentrations for a plurality of gas types whose components and concentrations are known; a detection unit that detects responses of the gas sensitive members of the three or more gas sensors for gas to be measured; and a gas type identification unit that calculates conversion concentrations of the plurality of gas types using the information stored in the storage unit and the responses detected by the detection unit and that identifies a gas type having a smallest variation among differences between conversion concentrations of a specific gas sensor of the three or more gas sensors and conversion concentrations corresponding to the other two or more gas sensors”.

In such a gas detection system, it is necessary to individually customize gas components to be detected. However, it is required not to customize an odor sensor depending on an environment in order to improve versatility of the device.

### SUMMARY OF THE INVENTION

The invention has been made in view of such a circumstance, and an object of the invention is to provide a gas detection system or the like that does not require customization of an odor sensor depending on an environment.

To solve the above problem, the invention provides a gas detection system. The gas detection system includes an element array including a plurality of gas detection elements having different characteristic sensitivities, a conversion unit configured to convert each of output values acquired from the gas detection elements constituting the element array into an output ratio as a ratio of the output value to a predetermined value, and a determination unit configured to determine, by comparing a normal range generated based on a result of executing first pattern classification on a past output ratio acquired from each of the gas detection elements constituting the element array in the past with the output ratio obtained by the conversion unit executing the conversion, whether the output ratio is within the normal range.

According to the invention, it is possible to provide a gas detection system or the like that does not require customization of an odor sensor depending on an environment.

Other problems, configurations, and effects will be clarified based on description of embodiments as follows.

### BRIEF DESCRIPTION OF THE DRAWINGS

FIG. 1 is a diagram showing a configuration of an odor measurement system according to the present embodiment;

FIG. 2 is a diagram showing a configuration example of a sensor array according to the present embodiment;

FIG. 3 is a diagram showing an example of an odor measurement device according to the present embodiment;

FIG. 4 is a diagram showing a procedure of first base correction according to the present embodiment;

FIG. 5 is a diagram showing a result of executing the first base correction on a sensor output in outside air;

FIG. 6 is a flowchart showing a processing procedure of pre-learning processing according to the present embodiment;

FIG. 7 is a diagram showing a result of measuring environmental gas for a certain period of time using the sensor array on which the first base correction has been executed;

FIG. 8 is a diagram showing output ratios of sensors;

FIG. 9 is a flowchart showing a procedure of measurement and analysis according to the present embodiment (part 1);

FIG. 10 is the flowchart showing the procedure of measurement and analysis according to the present embodiment (part 2);

FIG. 11 is a diagram showing time variation in sensor outputs in measurement;

FIG. 12 is a diagram showing the time variation in sensor outputs after second base correction;

FIG. 13 is a diagram showing a time change in output ratios;

FIG. 14 is a diagram showing a change in level;

FIG. 15 is a diagram showing a correspondence relationship between a cluster level and the number of operating devices in a factory;

FIG. 16 is a diagram showing an example of normal ranges;

FIG. 17 is an odor measurement result in an office; and

FIG. 18 is a diagram showing a hardware configuration of a cloud server.

### DESCRIPTION OF THE PREFERRED EMBODIMENTS

An embodiment according to the invention will be described hereinafter with reference to the accompanying drawings. The embodiment is an example for describing the invention, and omission and simplification are appropriately made for clarified description. The invention can be implemented in various other forms. Unless otherwise specified, components may be singular or plural.

Positions, sizes, shapes, ranges, and the like of the components showed in the drawings may not represent actual positions, sizes, shapes, ranges, and the like in order to facilitate understanding of the invention. Therefore, the invention is not necessarily limited to the positions, sizes, shapes, ranges, and the like disclosed in the drawings.

When there are a plurality of components having the same or similar functions, different subscripts may be added to the same reference numeral. When it is not necessary to distinguish the plurality of components from one another, the subscripts may be omitted in the description.

In the embodiment, processing executed by executing a program may be described. Here, a computer executes a program by a processor (for example, a CPU 212 (see FIG. 18) or a GPU), and executes processing determined by the program while using a storage resource (for example, a memory 211 (see FIG. 18)), an interface device (for example, a communication port), or the like. Therefore, a processor may be a subject of the processing executed by executing a program. Similarly, a subject of the processing executed by executing a program may be a controller, a device, a system, a computer, or a node. The controller, the device, the system, the computer, or the node includes a processor. The subject of the processing executed by executing a program may be a calculation unit, and may include a dedicated circuit that executes specific processing. Here, the dedicated circuit refers to, for example, a field programmable gate array (FPGA), an application specific integrated circuit (ASIC), and a complex programmable logic device (CPLD).

A program may be installed in a computer from a program source. The program source may be, for example, a program distribution server or a storage medium readable by a computer. When the program source is a program distribution server, the program distribution server may include a processor and a storage resource that stores a program to be distributed, and the processor of the program distribution server may distribute the program to be distributed to another computer. In the embodiment, two or more programs may be implemented as one program, or one program may be implemented as two or more programs.

### Odor Measurement System F

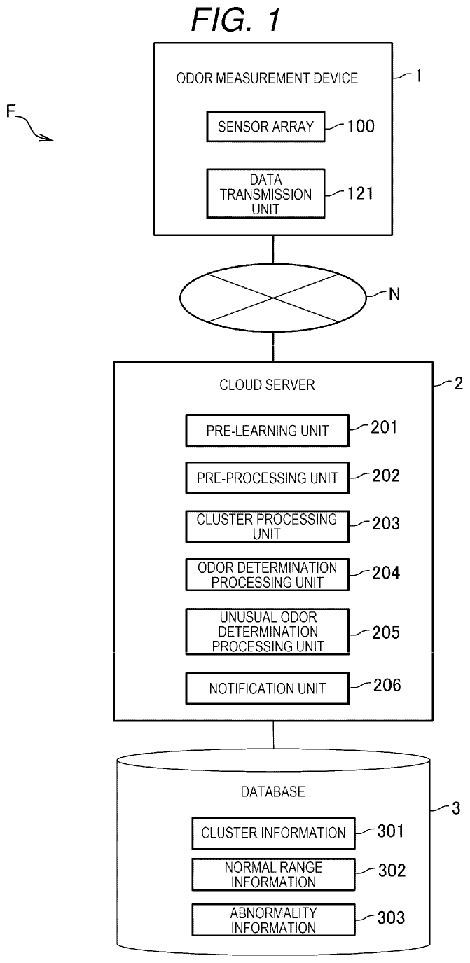

FIG. 1 is a diagram showing a configuration of an odor measurement system F according to the present embodiment.

As shown in FIG. 1, the odor measurement system F includes an odor measurement device 1, and a cloud server 2 connected to a database 3. The odor measurement device 1 and the cloud server 2 are connected to each other via a network N such as a wide area network (WAN). With such a configuration, the cloud server 2 provides a cloud service for a user of the odor measurement device 1. Although the cloud server 2 provides a cloud service in the present embodiment, the configuration of the odor measurement system F is not limited to the configuration shown in FIG. 1, and the cloud server 2 may be an in-house server or a personal computer (PC) owned by a person. The odor storage device 1 may include a storage resource (not shown) capable of storing data.

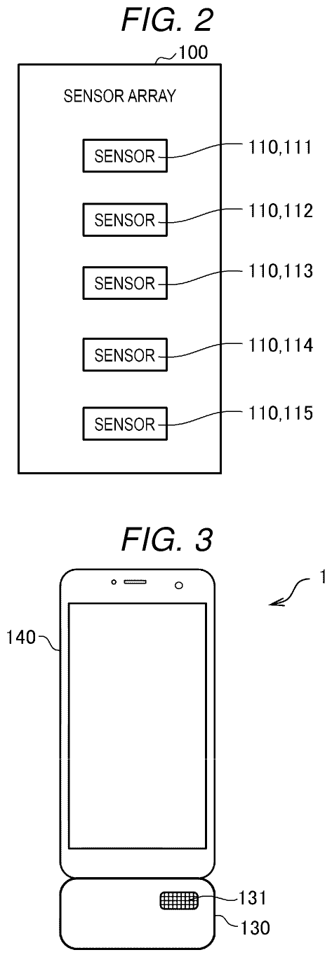

The odor measurement device 1 includes a sensor array 100 including a plurality of sensors 110 (see FIG. 2) that detect an odor component and that output the odor component as a signal, and a data transmission unit 121 that transmits the signal output by the sensor array 100 to the cloud server 2 as a sensor output. The sensor array 100 will be described later.

The cloud server 2 includes a pre-learning unit 201, a pre-processing unit 202, a cluster processing unit 203, an odor determination processing unit 204, an unusual odor determination processing unit 205, and a notification unit 206.

The pre-learning unit 201 converts a sensor output (output voltage) measured in advance by the sensor array 100 into an output ratio. Further, the pre-learning unit 201 classifies the sensor output of the sensors 110 into clusters by executing cluster analysis.

The preprocessing unit 202 executes pre-processing such as converting a measured sensor output of each sensor 110 into an output ratio to attain a format suitable for cluster classification to be executed thereafter. In the present embodiment, measurement in a processing stage of the pre-learning unit 201 is referred to as “pre-measurement”, and measurement in processing executed by the pre-processing unit 202, the cluster processing unit 203, the odor determination processing unit 204, the unusual odor determination processing unit 205, and the notification unit 206 is simply referred to as “measurement”.

The cluster processing unit 203 executes cluster classification based on the pre-processed sensor output. Specifically, the cluster processing unit 203 determines to which cluster the sensor output is classified among the clusters classified by the pre-learning unit 201. When a measured result coincides with unusual odor information, cluster analysis of the output ratio is executed again.

The odor determination processing unit 204 sets a level to a cluster, determines which level an odor of an environment to be measured is, and executes processing according to the level.

The unusual odor determination processing unit 205 determines whether an unusual odor has been detected based on the output ratio.

The notification unit 206 issues an alarm when the unusual odor determination processing unit 205 determines that an unusual odor has been detected.

The database 3 stores cluster information 301, normal range information 302, and abnormality information 303.

In the cluster information 301, information on a cluster generated by category analysis is stored.

The normal range information 302 stores a range of an output ratio determined to be normal by the unusual odor determination processing unit 205.

The abnormality information 303 stores information on an output ratio determined to be abnormal.

The network N may be capable of fifth-generation (5G) communication.

### Sensor Array 100

FIG. 2 is a diagram showing a configuration example of the sensor array 100 according to the present embodiment.

As shown in FIG. 2, the sensor array 100 includes the plurality of (five in the example in FIG. 2) sensors 110 (111 to 115).

In the present embodiment, the sensors 110 are odor sensors. Specifically, the sensors 110 are implemented by a semiconductor gas sensor, a sensitive film using an organic film, or the like, and respond to a specific odor. The sensors 111 to 115 are odor sensors having different characteristic sensitivities. For example, the sensor 111 is an odor sensor having a characteristic sensitivity for an ethanol-based odor, and the sensor 112 is an odor sensor having a characteristic sensitivity for a ketone-based odor. Similarly, a sensor 113 is an odor sensor having a characteristic sensitivity for a hydrogen-based odor, a sensor 114 is an odor sensor having a characteristic sensitivity for an ammonia-based odor, and a sensor 115 is an odor sensor having a characteristic sensitivity for a hydrocarbon-based odor.

The present embodiment shows an example in which five types of five sensors 110 are provided in the sensor array 100. However, the invention is not limited thereto, and two or more sensors 110 may be provided in the sensor array 100. Types of the sensors 110 provided in the sensor array 100 are different.

### Odor Measurement Device 1

FIG. 3 is a diagram showing an example of the odor measurement device 1 according to the present embodiment.

As shown in FIG. 3, in the odor measurement device 1, an odor measurement terminal 130 is connected to a smartphone 140. Here, the odor measurement terminal 130 is connected to the smartphone 140 via a universal serial bus (USB) or the like. Here, the odor measurement terminal 130 includes the sensor array 100 (see FIGS. 1 and 2). As shown in FIG. 3, the odor measurement terminal 130 has an opening 131, and each of the sensors 110 of the sensor array 100 shown in FIG. 2 detects odor molecules introduced into the odor measurement terminal 130 through the opening 131. In order to take in the odor molecules, an intake fan or the like may be provided in an exhaust port (not shown), or exhaust may be performed by a pump (not shown) or the like.

In the example shown in FIG. 3, the smartphone 140 corresponds to the data transmission unit 121 in FIG. 1.

In the present embodiment, the smartphone 140 is connected to the odor measurement terminal 130, but the invention is not limited thereto. The odor measurement terminal 130 may be one device including the sensor array 100 and the data transmission unit 121.

### First Base Correction

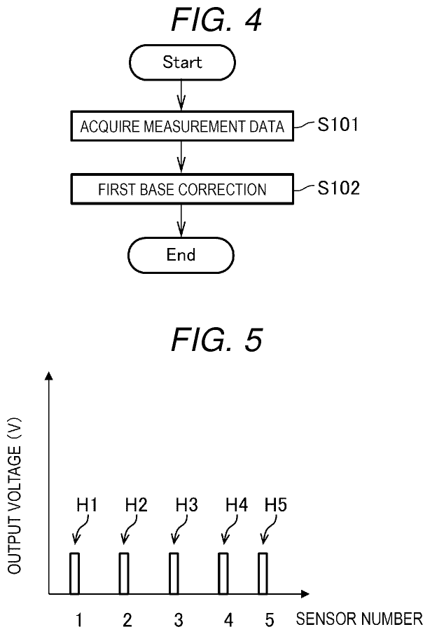

Hereinafter, the first base correction in the present embodiment will be described with reference to FIGS. 4 and 5. FIGS. 1 and 2 will be referred to appropriately. Hereinafter, a case will be described in which an environment to be measured is a factory.

FIG. 4 is a diagram showing a procedure of the first base correction according to the present embodiment.

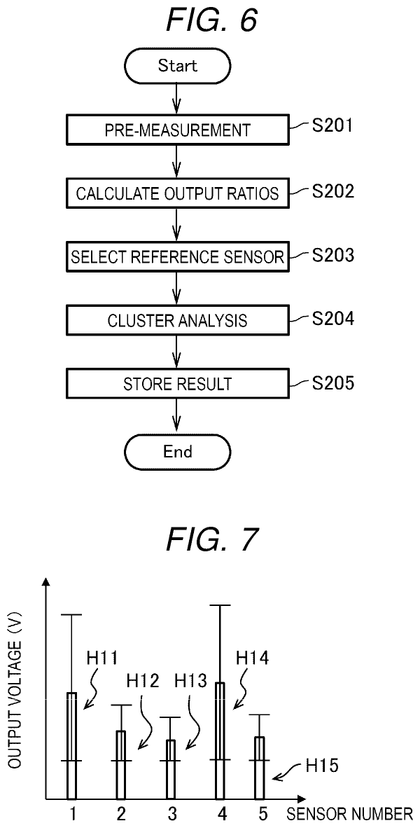

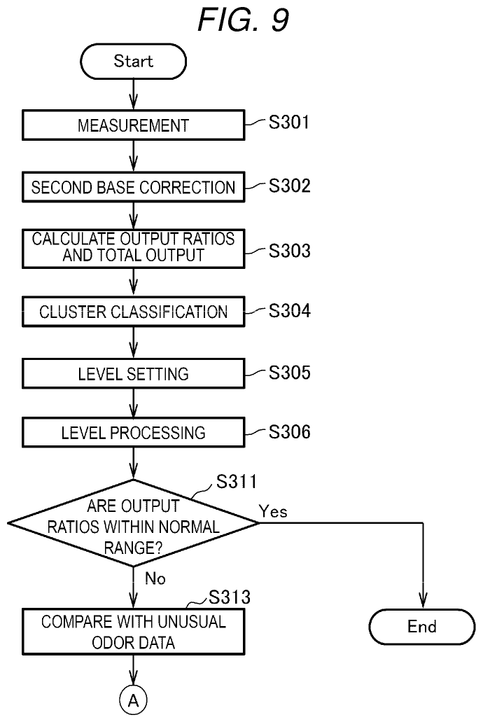

First, the pre-learning unit 201 acquires sensor outputs in the outside air (base environment) from the sensors 110 (S101). Measurement executed in step S101 is different from pre-measurement in step S201 in FIG. 6 to be described later and measurement in step S301 in FIG. 9 to be described later. The sensor outputs include output signals (signals of output voltages) output from the sensors 110. In an example according to the present embodiment, the outside air refers to air outside the factory.

Then, the pre-learning unit 201 executes correction (first base correction) such that each sensor output becomes constant (S102). In the first base correction, the sensors 110 are adjusted individually such that the sensor outputs of the sensors 110 are substantially equal to one another. Adjustment is generally executed by the user.

The measurement in the outside air in step S101 may be executed for a certain period of time, and the pre-learning unit 201 may execute the first base correction based on an average value of the measurement.

First, FIG. 5 is a diagram showing a result of executing the first base correction on the sensor outputs in the outside air. In the example according to the present embodiment, the outside air is air outside the factory.

In FIG. 5, a vertical axis represents an output voltage (V) of the sensor 110, and a horizontal axis represents a sensor number. As described above, the sensor outputs include the signals of the output voltages in the sensors 110.

In FIG. 5, a sensor number “1” indicates the sensor 111, a sensor number “2” indicates the sensor 112, and a sensor number “3” indicates the sensor 113. Similarly, a sensor number “4” indicates the sensor 114, and a sensor number “5” indicates the sensor 115. The same applies to sensor numbers in FIGS. 7, 8, 16, and 17 to be described later.

That is, in FIG. 5, “H1” indicates an output voltage (V) of the sensor 111, and “H2” indicates an output voltage of the sensor 112. Similarly, in FIG. 5, “H3” indicates an output voltage of the sensor 113, “H4” indicates an output voltage of the sensor 114, and “H5” indicates an output voltage of the sensor 115. As described above, the sensors 110 are adjusted individually by the user such that the output voltages indicated by “H1” to “H5” are substantially equal to one another.

### Pre-Learning Processing

Next, the pre-learning processing according to the present embodiment will be described with reference to FIGS. 6 to 8. FIGS. 1 and 2 will be referred to appropriately.

FIG. 6 is a flowchart showing a processing procedure of the pre-learning processing according to the present embodiment.

First, the pre-learning unit 201 executes, using the sensor array 100 on which the first base correction has been executed, pre-measurement for a certain period of time in an environment (factory in the present embodiment) to be measured (S201 in FIG. 6).

FIG. 7 is a diagram showing a result obtained by executing pre-measurement of a factory for a certain period of time (for example, one day) using the sensor array 100 on which the first base correction has been executed.

A vertical axis and a horizontal axis in FIG. 7 are the same as those in FIG. 5, and thus description thereof will be omitted.

In a bar graph in FIG. 7, a bar indicates an average value of the output voltages of the sensors 110 in a pre-measurement time. Whiskers attached to the bars indicate a variation range of the output voltages in the pre-measurement time. That is, an upper whisker indicates a maximum value of the output voltages in the pre-measurement time, and a lower whisker indicates a minimum value of the output voltages in the pre-measurement time.

In FIG. 7, “H11” indicates an output voltage (V) of the sensor 111, and “H12” indicates an output voltage of the sensor 112. Similarly, in FIG. 7, “H13” indicates an output voltage of the sensor 113, “H14” indicates an output voltage of the sensor 114, and “H15” indicates an output voltage of the sensor 115.

Then, the pre-learning unit 201 calculates output ratios of the sensors 110 based on the result shown in FIG. 7 (S202 in FIG. 6). In step S202, the pre-learning unit 201 calculates the output ratios for each time.

An output ratio Yn of the certain sensor 110 at a certain time t is calculated according to the following equations (1) and (2).

Yn(t)=|Xn(t)|/Xall(t) (1)

Xall(t)=|X1(t)|+|X2(t)|+|X3(t)|+|X4(t)|+|X5(t)| (2)

Here, n is a sensor number. That is, when n=1, the sensor 111 is indicated, and when n=2, the sensor 112 is indicated. Similarly, when n=3, the sensor 113 is indicated, when n=4, the sensor 114 is indicated, and when n=5, the sensor 115 is indicated. X1(t) indicates the output voltage of the sensor 111 at the time t, and X2(t) indicates the output voltage of the sensor 112 at the time t. Similarly, X3(t) indicates the output voltage of the sensor 113 at the time t, X4(t) indicates the output voltage of the sensor 114 at the time t, and X5(t) indicates the output voltage of the sensor 115 at the time t. Since the output voltages may be shifted to a negative side, each output voltage is represented by an absolute value.

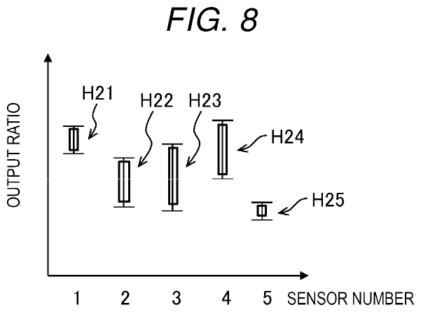

FIG. 8 is a diagram showing output ratios of the sensors 110.

In FIG. 8, a vertical axis represents the output ratios, and a horizontal axis represents sensor numbers. The sensor numbers are the same as those in FIG. 5.

In FIG. 8, “H21” indicates the output ratio of the sensor 111, and “H22” indicates the output ratio of the sensor 112. Similarly, in FIG. 8, “H23” indicates the output ratio of the sensor 113, “H24” indicates the output ratio of the sensor 114, and “H25” indicates the output ratio of the sensor 115. In FIG. 8, a box indicates a variation in output ratio in the pre-measurement time. An error bar indicates a range of ±2σ with respect to an average value of the output ratios. Here, σ is a standard deviation.

As described above, an output ratio represented in the equations (1) and (2) is calculated for each pre-measurement time of the sensor array 100. A variation region of the output ratio is indicated by the box in FIG. 8.

Then, the pre-learning unit 201 selects a reference sensor from the sensors 110 (S203 in FIG. 6).

The reference sensor is selected according to the following conditions (A1) and (A2).

(A1) The reference sensor belongs to a category having a small variation range (whiskers in FIG. 7) of the output voltages shown in FIG. 7. The category having a small variation range means that the variation range of the output voltages is equal to or less than a predetermined value.

(A2) A variation range (box in FIG. 8) of the output ratios is maximum.

The pre-learning unit 201 selects the reference sensor by prioritizing the condition (A2) over the condition (A1). For example, when variation ranges of the output ratios of the sensors 110 constituting the sensor array 100 are approximately the same, the pre-learning unit 201 selects the reference sensor based on the condition (A1). A fact that the variation ranges of the output ratios of the sensors 110 are approximately the same is described below. For example, a variation range of the sensor 110 having a maximum variation range is denoted by Wmax. A variation range of the sensor 110 other than the sensor 110 having a maximum variation range is denoted by W. In this case, Wmax>W≥0.95×Wmax. In examples in FIGS. 7 and 8, the sensor number “3”, that is, the sensor 113 is selected as the reference sensor.

Then, the pre-learning unit 201 executes the cluster analysis (first pattern classification) based on the output ratios of the sensors 110 (S204 in FIG. 6). A cluster is a set of groups of the output ratios of the sensors 110.

For example, when the output ratios of the sensors 111 to 115 are {x1, x2, x3, x4, x5}, a group of the output ratios is classified into a “cluster C1”. When the output ratios of the sensors 111 to 115 are {x11, x21, x31, x41, x51}, a group of the output ratios is classified into a “cluster C2” different from the “cluster C1”. In this way, the cluster is a set of groups of values of the sensors 111 to 115.

Sensitivity measurement of the sensors 110 for an environment to be measured is executed by processing in steps S201 to S203.

The pre-learning unit 201 stores a result of the cluster analysis in step S204 in FIG. 6 into the database 3 as the cluster information 301 (S205).

### Measurement and Analysis

Next, the measurement and analysis according to the present embodiment will be described with reference to FIGS. 9 to 16. FIGS. 1 and 2 will be referred to appropriately.

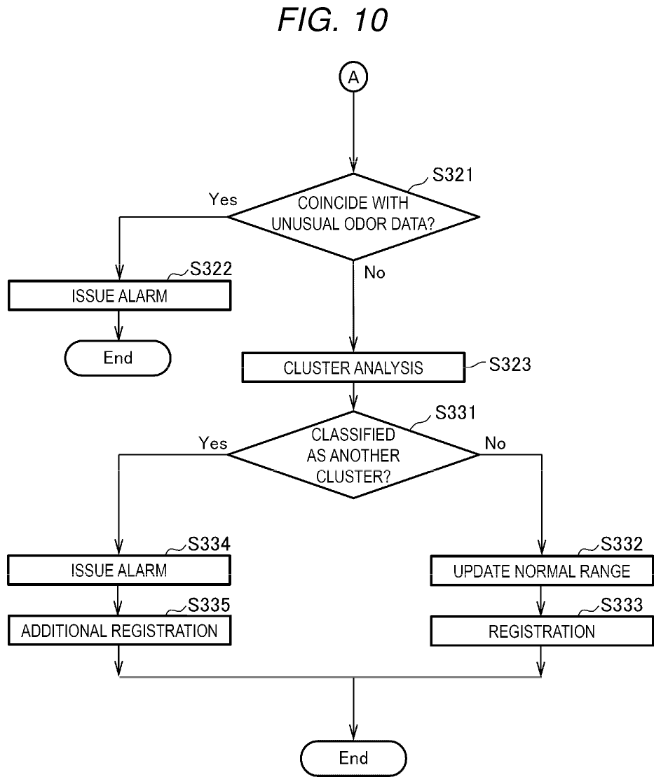

FIGS. 9 and 10 are a flowchart showing a procedure of the measurement and analysis according to the present embodiment.

First, the sensor array 100 executes real-time measurement (measurement), for example, at an interval of 0.5 seconds in an environment (factory in the present embodiment) to be measured (S301 in FIG. 9).

Subsequently, the pre-processing unit 202 executes second base correction (S302 in FIG. 9).

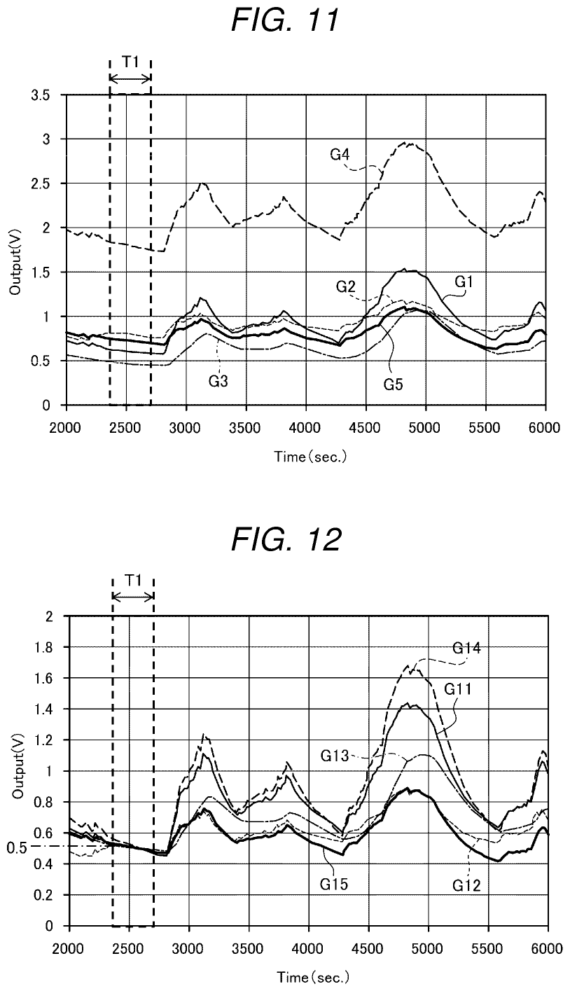

The second base correction executed in step S302 in FIG. 9 will be described with reference to FIGS. 11 and 12.

FIG. 11 is a diagram showing a time variation of sensor outputs in measurement.

In FIG. 11, graphs G1 to G5 indicate time variations of the sensor outputs (output voltages) of the sensors 110.

Here, the graph G1 indicates the time variation of the output voltage of the sensor 111, the graph G2 indicates the time variation of the output voltage of the sensor 112, and the graph G3 indicates the time variation of the output voltage of the sensor 113. Further, the graph G4 indicates the time variation of the output voltage of the sensor 114, and the graph G5 indicates the time variation of the output voltage of the sensor 115.

In FIG. 11, a period T1 is a blank measurement period. In the blank measurement period, it is considered that an odor is not detected as much as possible in an environment to be measured. For example, when the environment to be measured is a factory, a rest time is considered as the blank measurement period T1. The blank measurement period may be a period during the measurement executed in step S301 in FIG. 9, or may be a period during other measurement different from the measurement executed in step S301.

The pre-processing unit 202 executes the second base correction shown in step S302 in FIG. 9 by aligning the sensor outputs of the sensors 110 with a reference output for the outputs of the sensors 110 shown in FIG. 11. The reference output is an average value of the reference sensor in the blank measurement period. As described above, the sensor 113 is selected as the reference sensor in pre-learning in the present embodiment. In an example in FIG. 11, since an average value of the sensor 113 (graph G3) in the blank measurement period T1 is “0.5 V”, the sensor outputs of the sensors 110 are aligned with “0.5 V” in the blank measurement period T1. Here, an example has been described in which voltages are aligned with 0.5 V. Alternatively, a reference for alignment may be freely determined in consideration of a variation range of a target environment.

FIG. 12 shows a result (that is, normalized result) obtained by aligning the sensor outputs (output voltages) of the sensors 110 with “0.5V” for the sensor outputs in FIG. 11.

In FIG. 12, a graph G11 indicates a time variation of the output voltages after the second base correction of the sensor 111, and a graph G12 indicates a time variation of the output voltages after the second base correction of the sensor 112. A graph G13 indicates a time variation of the output voltages after the second base correction of the sensor 113, a graph G14 indicates a time variation of the output voltages after the second base correction of the sensor 114, and a graph G15 indicates a time variation of the output voltages after the second base correction of the sensor 115.

In FIG. 12, the period T1 is a blank measurement period as in FIG. 11.

In the second base correction, a time may be determined at which the sensor outputs of the sensors 110 are aligned. In an example in FIG. 12, the sensor outputs of the sensors 110 are aligned at a central time of the blank measurement period T1.

Thereafter, the pre-processing unit 202 substitutes the sensor outputs after the second base correction into the equations (1) and (2), and calculates output ratios at each measurement time and a total output (equation (2)) (S303 in FIG. 9). The pre-processing unit 202 may not necessarily calculate both the output ratios and the total output in step S303. When the output ratios are used for a level to be described later, the pre-processing unit 202 may calculate the output ratios in step S303. When the total output is used for the level, the pre-processing unit 202 may calculate the total output in step S303.

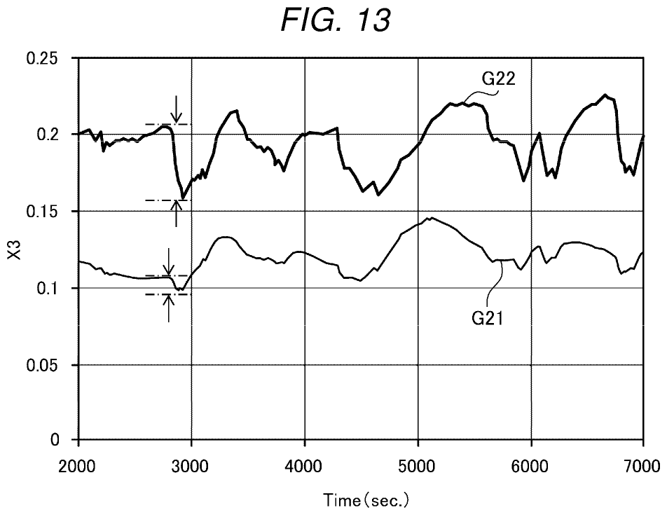

FIG. 13 is a diagram showing a time change in output ratios calculated in step S303.

A graph G21 is a graph showing a time change in output ratios of the sensor 113 after the second base correction has been executed. A graph G22 is a graph shown as a comparison target, and is a graph showing a time change in the output ratios of the sensor 113 before the second base correction is executed.

Widths of portions indicated by dash-dotted lines in FIG. 13 indicate variation ranges of the graphs G21 and G22 at the same time. When widths of dash-dotted lines are compared, a variation range of the output ratios in the graph G12 (after the second base correction) is larger than that in the graph G11 (before the second base correction). That is, after the sensor outputs have been normalized by executing the second base correction, the output ratios are calculated, so that SN ratios of the sensor outputs having a small variation range can be increased.

After step S303 in FIG. 9, the cluster processing unit 203 executes cluster classification using calculated output ratios (S304 in FIG. 9). In step S304, the cluster processing unit 203 determines to which cluster a group of the output ratios of the sensors 110 at each time belongs. A cluster is a result of the cluster analysis executed by the pre-learning unit 201 in step S204 in FIG. 6. In the present embodiment, the number of clusters is “5”. Alternatively, the number of clusters may be “2” or more.

### Level Setting

After step S304 in FIG. 9, the odor determination processing unit 204 sets a level to each cluster (S305 in FIG. 9).

A level setting method includes, for example, the following methods.

(B1) The level is set according to an average value of the output ratios of the reference sensor (the sensor 113 in the present embodiment). The average value in (B1) refers to an average value of the output ratios in a cluster.

For example, the following levels are set for average values in the clusters of the sensor 113 selected as the reference sensor in the example according to the present embodiment.

Average value: 0.16→Level 5

Average value: 0.17→Level 4

Average value: 0.19→Level 3

Average value: 0.21→Level 2

Average value: 0.22→Level 1

Here, a reason why the level increases as the average value decreases is that the sensor 113 is the sensor 110 whose sensor output decreases as a predetermined odor is stronger. The sensor 113 is a calculation target of the average value. When the sensor 110 whose sensor output increases as the predetermined odor is stronger is selected as the reference sensor, the level may increase as the average value increases. The predetermined odor refers to an oily smell in the example according to the present embodiment.

(B2) The level is set according to an average value of Xall (equation (2)) in a cluster. Hereinafter, the average value of Xall in a cluster is referred to as a total average value. For example, the odor determination processing unit 204 calculates the total average value of all the sensors 110 in each cluster. Then, the odor determination processing unit 204 sets a level for each cluster based on the calculated total average value. For example, levels are set as follows.

Total average value: 5.5 V→Level 5

Total average value: 4.2 V→Level 4

Total average value: 3.7 V→Level 3

Total average value: 3.4 V→Level 2

Total average value: 3.1 V→Level 1

In the above example, the level increases as the total average value increases. However, for example, when the total average value decreases as the predetermined odor is stronger, the level may increase as the total average value decreases.

In the present embodiment, a level of a cluster is set based on an average value, but the invention is not limited thereto. For example, in a case in which an average value is not suitable as a level setting reference, such as a case in which a large outlier is present, the odor determination processing unit 204 may set the level of the cluster according to a median value.

In the present embodiment, the level of the cluster is set at a stage of step S305 in FIG. 9. Alternatively, the pre-learning unit 201 may set the level of the cluster after step S204 in FIG. 6.

Both of the methods (B1) and (B2) may reverse an increasing direction of the level.

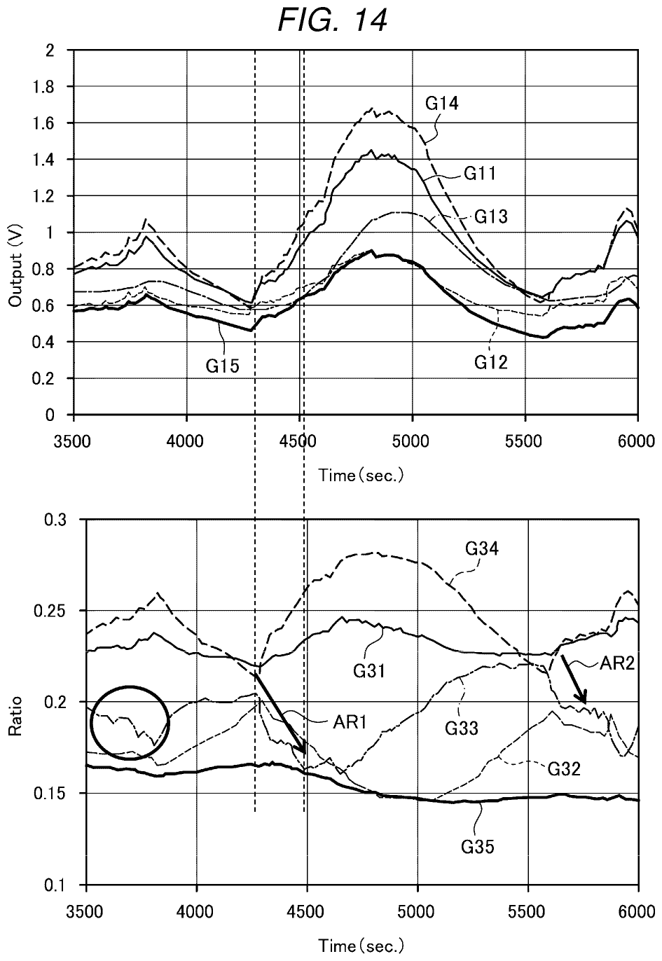

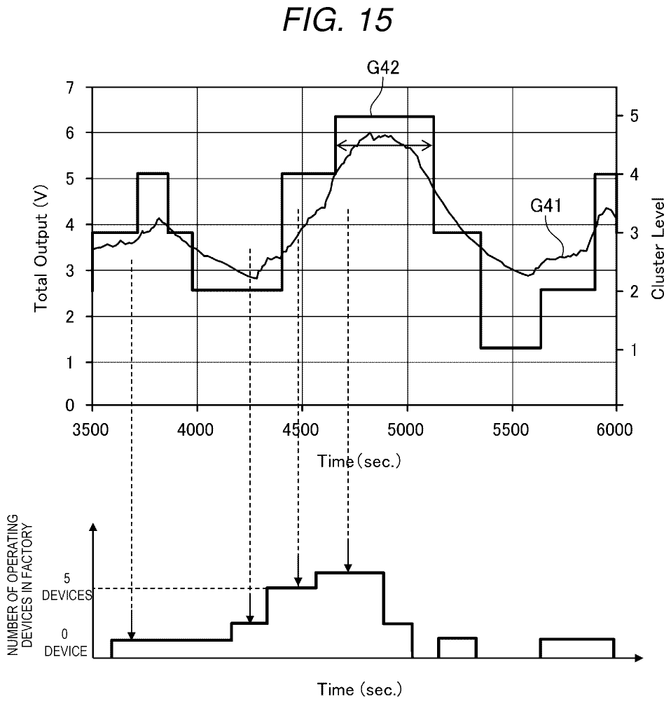

FIG. 14 is a diagram showing a change in level.

An example of the level set according to the method (B1) will be described with reference to FIG. 14.

An upper part of FIG. 14 is a diagram showing the sensor outputs of the sensors 110 after the second base correction. The upper part of FIG. 14 is obtained by extracting a portion of times “3500” to “6000” in FIG. 12. Graphs G11 to G15 in FIG. 14 are the same as those in FIG. 12, and thus the description thereof will be omitted.

A lower part of FIG. 14 shows a time change in the output ratios calculated according to the method shown in the equations (1) and (2) based on the sensor outputs after the second base correction shown in the upper part of FIG. 14. Times in the upper part and the lower part of FIG. 14 coincide with each other.

In the lower part of FIG. 14, a graph G31 indicates a time variation of the output ratios of the sensor 111, a graph G32 indicates a time variation of the output ratios of the sensor 112, a graph G33 indicates a time variation of the output ratios of the sensor 113, a graph G34 indicates a time variation of the output ratios of the sensor 114, and a graph G35 indicates a time variation of the output ratios of the sensor 115.

In the lower part of FIG. 14, a portion indicated by a solid-line circle indicates an odor increase timing, and the output ratio of the sensor 113 indicated by the graph G33 decreases according to the odor increase timing. This indicates that the output ratio of the sensor 113 decreases due to a component contained in oil.

Then, the output ratio of the sensor 113 greatly decreases at positions indicated by arrows AR1 and AR2 in the lower part of FIG. 14. That is, when the level set according to the method (B1) is taken as an example, the level changes from the “level 2” to the “level 4” at the position of the arrow AR1. Similarly, the level changes from “level 1” to “level 3” at the position of the arrow AR2. Vertical dashed lines shown from the upper part to the lower part of FIG. 14 indicate a period in which a value of the graph G33 is greatly decreased near the arrow AR1.

After step S305 in FIG. 9, the odor determination processing unit 204 executes level determination and executes processing (level processing) according to a result of the level determination (S306 in FIG. 9). By executing the second base correction as described above, the SN ratios of the output ratios are improved, so that the change in the level is remarkably shown. That is, the odor determination processing unit 204 can easily execute the level determination by executing the second base correction.

As an example of the level processing, the following can be considered.

(D1) Air conditioning management in the factory according to the level.

(D2) Estimation and abnormality detection of the number of operating devices in the factory.

(D3) Unusual odor leakage warning from the factory.

If unusual odor leakage is in, for example, the “level 4” or higher, the odor determination processing unit 204 determines that the unusual odor leakage occurs.

Hereinafter, a specific example of (D2) will be described with reference to FIG. 15.

FIG. 15 is a diagram showing a correspondence relationship between a cluster level and the number of operating devices in the factory.

In an upper part of FIG. 15, a graph G41 indicates a time change of total sensor outputs (corresponding to the equation (2)), and a graph G42 indicates a change in cluster level. FIG. 15 shows an example in which the level is set according to the method (B2). That is, FIG. 15 shows an example in which the level is set according to a total average value (that is, the method (B2)) of all the sensors 110 in a cluster. In the graph G42, portions horizontal to a horizontal axis correspond to the clusters. In this regard, in the upper part of FIG. 15, a vertical axis on a left side of the drawing shows a total average value corresponding to the graph G41, and a vertical axis on a right side of the drawing shows a level of the cluster corresponding to the graph G42.

A lower part of FIG. 15 shows a time change in the number of operating devices in the factory at a time corresponding to the graphs shown in the upper part of FIG. 15. The number of operating units in the factory is counted based on operation records of the devices in the factory. In the following description, the “number of operating devices” refers to the number of the operating devices in the factory.

Data shown in the upper part and the lower part of FIG. 15 are measured in advance, and the clusters and the number of operating devices in the factory are associated with each other by an administrator as indicated by dashed-line arrows shown in FIG. 15. By such association, the odor determination processing unit 204 can estimate the number of operating devices in the factory based on the clusters. As shown in FIG. 15, the clusters do not necessarily correspond to the number of operating devices in the factory on an exact one-to-one basis. In this case, the odor determination processing unit 204 may associate the clusters with the number of operating devices in the factory according to, for example, the number of operating devices in the factory weighted by a time when each cluster overlaps with the number of operating devices. If a deviation occurs between the actual number of operating devices and the estimated number of operating devices based on the clusters, it is considered that an abnormality in the devices in the factory, a change in loads on the devices in the factory, or the like has an influence. When the number of operating devices in the factory is estimated as in the (D2) described above, the odor determination processing unit 204 executes processing based on the level and the number of operating devices. The level and the number of operating devices are associated according to the methods described above.

Then, the odor determination processing unit 204 estimates the number of operating devices in the factory based on the determined level. Thereafter, the odor determination processing unit 204 determines whether a deviation occurs between the estimated number of operating devices in the factory and the actual number of operating devices, and detects an abnormality in the devices in the factory and loads on the devices in the factory ((D2) described above).

### Unusual Odor Determination

Subsequently, the unusual odor determination processing unit 205 reads the normal range information 302 from the database 3, and determines whether the output ratios of the sensors 110 are within a normal range (S311).

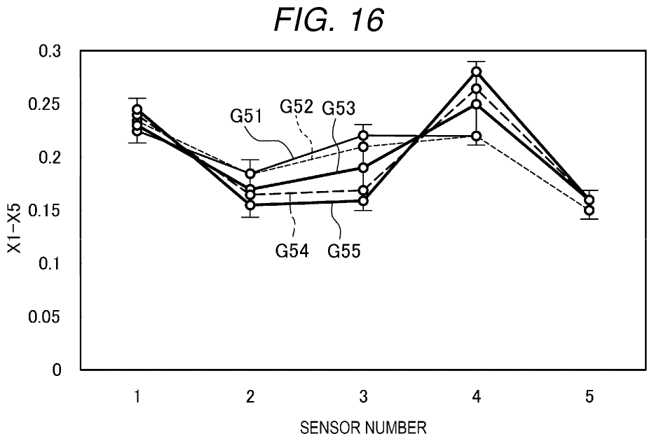

FIG. 16 is a diagram showing an example of the normal range. Here, the normal range means a range in which it is determined that no unusual odor is generated.

In FIG. 16, each graph indicates a cluster. For example, a graph G51 indicates the “cluster C1”, a graph G52 indicates the “cluster C2”, and a graph G53 indicates a “cluster C3”. Similarly, a graph G54 indicates a “cluster C4”, and a graph G55 indicates a “cluster C5”. Graphs G51 to G55 show a result of connecting the average values of the output ratios in the clusters. A horizontal axis indicates a sensor number.

In FIG. 16, whiskers indicate a normal range of the output ratios of the sensors 110. The normal range is a range of the output ratios included in the clusters (the “cluster C1” to the “cluster C5” in the example according to the present embodiment) for the sensors 110. In FIG. 16, a normal range of the sensor 113 (sensor number “3”) selected as the reference sensor is the largest.

The normal ranges as shown in FIG. 16 are set in advance. If the unusual odor determination processing unit 205 determines that the output ratio of any one of the sensors 110 among the current output ratios falls within the normal range (S311→Yes in FIG. 9), the cloud server 2 ends the processing.

If the output ratio of any one of the sensors 110 among the current output ratios deviates from the normal range (S311→No in FIG. 9), the unusual odor determination processing unit 205 compares the abnormality information 303 stored in the database 3 with the current output ratios (S313 in FIG. 9). In the abnormality information 303, information (the groups of the output ratios of the sensors 110) on the output ratio determined to be abnormal in the past is stored.

Then, the unusual odor determination processing unit 205 determines whether the current output ratios coincide with registered unusual odor data (S321 in FIG. 10).

Then, if the current output ratios coincide with the abnormality information 303 (S321→Yes in FIG. 10), the unusual odor determination processing unit 205 determines that an unusual odor has been detected, and the notification unit 206 issues an alarm (S322 in FIG. 10).

On the other hand, if the current output ratios do not coincide with the registered abnormality information 303 (S321→No), the cluster processing unit 203 executes the cluster analysis again using data of the output ratios including the output ratio determined to be deviated from the normal range in step S311 in FIG. 9 (S323 in FIG. 10: second pattern classification).

Then, as a result of the cluster analysis, the unusual odor determination processing unit 205 determines whether the output ratio deviating from the normal range is classified as a cluster (another cluster) different from the previous clusters (S331 in FIG. 10).

When the output ratio deviating from the normal range is not classified as a cluster different from the previous clusters (S331→No), the unusual odor determination processing unit 205 updates the normal range (S332 in FIG. 10) and registers the updated normal range into the database 3 as the normal range information 302 (S333 in FIG. 10). A fact that the output ratio deviating from the normal range is not classified as a cluster different from the previous clusters means that an odor is mistakenly detected as an unusual odor and the output ratio is included in the normal range. In such a case, the unusual odor determination processing unit 205 updates the normal range to the latest normal range in step S332.

When the output ratio deviating from the normal range is classified as a cluster different from the previous clusters (S331→Yes), the unusual odor determination processing unit 205 determines the output ratio determined to be deviated from the normal range as information indicating that an unusual odor has been detected. Then, the notification unit 206 issues an alarm (S334), and the unusual odor determination processing unit 205 additionally registers the output ratio determined to be deviated from the normal range as the abnormality information 303 (S335).

### Modification

In the present embodiment, odor measurement in the factory is described as an example. Alternatively, the odor measurement system F according to the present embodiment may be applied to odor measurement in another environment to be measured.

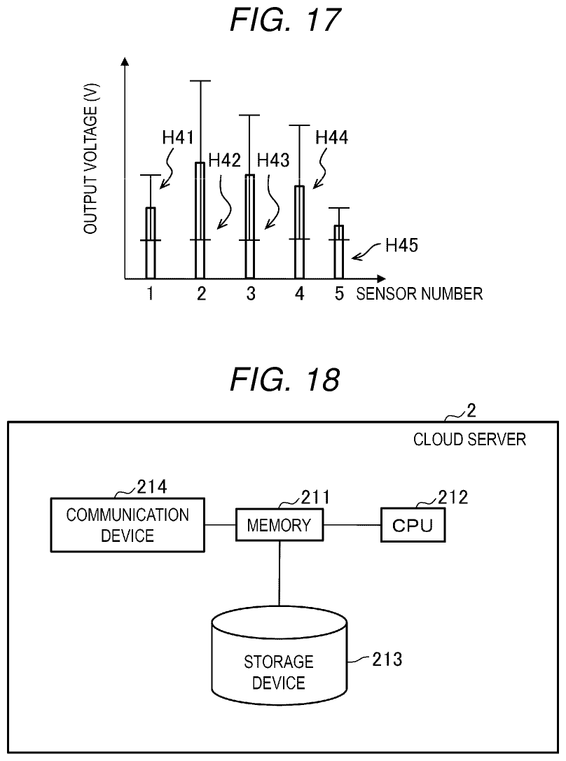

For example, FIG. 17 is a pre-measurement result in an office. FIG. 17 corresponds to FIG. 7. That is, in the office, FIG. 17 shows average values and variation ranges when pre-measurement is executed for a predetermined period at the time of pre-learning. In FIG. 17, “H41” to “H45” are the same as “H11” to “H15” in FIG. 7, respectively. The sensors 111 to 115 used in FIG. 17 are the same sensors 110 as the sensors 111 to 115 used in FIG. 7, respectively.

Here, when the result in FIG. 17 is used to select the reference sensor according to the above method, the sensor 115 (“H45”) is selected as the reference sensor. In this way, even when the sensor array 100 having the same configuration is used, the sensor 110 that varies depending on the environment to be measured is selected as the reference sensor. In other words, the odor measurement system F according to the present embodiment can use the sensor array 100 having the same configuration for various environments to be measured. That is, in the odor measurement system F, the reference sensor is selected for each environment to be measured. The cluster and the normal range are also set for each measurement mode environment by selecting the reference sensor for each environment to be measured.

The odor measurement system F according to the present embodiment can be applied to places other than the factory or the office described in the present embodiment. An application range of the odor measurement system F according to the present embodiment is wide, and for example, the odor measurement system F can be applied to odor management in a smoking area or odor management of a crop or soil.

In the present embodiment, the sensor 110 having the smallest variation range and a maximum output ratio range is selected as the reference sensor, but the method for selecting the reference sensor is not limited thereto.

In the present embodiment, the equations (1) and (2) are used as calculation expressions of an output ratio, but the invention is not limited thereto. For example, the output ratio may be calculated according to the following equation (11) in which an absolute value of the sensor output (output voltage) of each sensor 110 is divided by an absolute value of the sensor output of the reference sensor.

Yn(t)=|Xn(t)|/|Xc(t)| (11)

Here, n is the number of the sensor 110. Further, t is a time. Xc(t) is the sensor output of the reference sensor. For example, since the sensor 115 is selected as the reference sensor in the result in FIG. 17, Xc(t)=X5(t).

The output ratio as in the equation (11) may be used when the sensor 110, in which the sensor output of the sensor 110 changes with a logarithmic function, is used. Examples of the sensor 110, in which the sensor output of the sensor 110 changes with the logarithmic function, include an odor sensor using a crystal oscillator. Even when the sensor 110 in which the sensor output changes with the logarithmic function and the sensor 110 in which the sensor output changes linearly are mixed, the output ratio according to the equation (11) may be used.

Two or more reference sensors may be selected. Further, the quantitative sensor 110 may be used as a reference sensor. Therefore, the sensors 110 constituting the sensor array 100 may include a combination of the qualitative sensor 110 and the quantitative sensor 110. Examples of the quantitative odor sensor include a photoacoustic sensor and a near-infrared ray absorption sensor. Examples of the qualitative odor sensor include a semiconductor sensor and an odor sensor using a sensitive film having a chemical adsorption function.

### Hardware Configuration

FIG. 18 is a diagram showing a hardware configuration of the cloud server 2.

The cloud server 2 includes a memory 211, a central processing unit (CPU) 212, a storage device 213 such as a hard disk (HD), and a communication device 214.

Programs stored in the storage device 213 are loaded into the memory 211 and executed by the CPU 212, whereby the units 201 to 206 shown in FIG. 1 are implemented. The communication device 214 communicates with the odor measurement terminal 130 (see FIG. 3) via the network N (see FIG. 1).

The odor measurement system F according to the present embodiment includes the plurality of sensors 110 having different characteristic sensitivities. The odor measurement system F compares a normal range obtained as a result of executing cluster analysis on the output ratios based on the sensor outputs output in the past (pre-measurement) from the sensors 110 with the output ratios based on the sensor outputs currently output from the sensors 110. Then, as a result of the comparison, the odor measurement system F determines whether the output ratios based on the sensor outputs currently output from the sensors 110 are within the normal range. A sensor configuration that varies for each environment to be measured is not required using the normal range obtained as a result of executing the cluster analysis on the past output ratios, and thus versatility of the sensor 110 is improved. In other words, it is not required to customize the configuration of the sensor 110 of the sensor array 100 depending on the environment to be measured.

In related-art odor measurement techniques, drift or the like may occur in the output of the odor sensor due to environmental change during measurement or gas flow velocity change at the time of suctioning ambient gas by a pump. The odor measurement system F according to the present embodiment calculates the output ratio obtained by normalizing the sensor outputs of the sensors 110. Then, the odor measurement system F determines normality or abnormality using the output ratio. Accordingly, it is possible to correct a voltage change (drift) of the entire sensors 110 due to the environmental change during measurement. Therefore, it is possible to execute the category analysis in which an influence of drift has been corrected by executing the category analysis based on the output ratio. The normal range generated based on such category analysis is a normal range in which the influence of drift has been corrected. Thus, the odor measurement system F according to the present embodiment can improve accuracy of normality or abnormality determination.

Further, in the related-art odor measurement techniques, a suction completion type measurement method has been disclosed, which has problems that measurement time is long and real-time performance cannot be ensured. The odor measurement system F according to the present embodiment can ensure real-time performance by classifying the measured output ratios into categories generated in advance.

The odor measurement system F according to the present embodiment selects a reference sensor for each environment to be measured, and sets a normal range for each environment to be measured. Accordingly, the odor measurement system F according to the present embodiment can detect an odor, which is a non-examination target, as an unusual odor.

The odor measurement system F according to the present embodiment executes machine learning (cluster analysis) on the sensor output. Accordingly, a database of information on odors can be created, and a platform of odors can be implemented.

The odor measurement system F according to the present embodiment sets a level for a category, and executes processing according to the level. Accordingly, it is possible to appropriately execute processing according to a degree of an odor.

The odor measurement system F according to the present embodiment repeats the category analysis while accumulating data of the sensor outputs. In this way, the odor measurement system F can sequentially update the normal range, thereby improving accuracy of unusual odor determination. With repeated learning, the odor measurement system F may include the odor information that has been excluded from the normal range into the normal range, and the accuracy of unusual odor determination can be improved.

Further, as a result of the category analysis, when the current output ratio is classified into another category that does not belong to the previous categories, or when the current output ratio coincides with preset abnormality information, the notification unit 206 issues an alarm. Accordingly, it is possible to notify the user of odor abnormality detection.

In the present embodiment, an odor sensor is used as the sensor 110 to measure an odor, but the invention is not limited thereto. A gas sensor may be used as the sensor 110 to measure gas.

In the embodiments, control lines and information lines considered to be necessary for description are shown, and not all control lines and information lines are necessarily shown in a product. Actually, it may be considered that almost all the configurations are connected to one another.

The invention is not limited to the embodiments described above, and includes various modifications. For example, the above embodiments have been described in detail for easy understanding of the invention, and the invention is not necessarily limited to those having all the configurations described above. A part of a configuration according to one embodiment can be replaced with a configuration according to another embodiment, and a configuration according to one embodiment can be added to a configuration according to another embodiment. A part of the configuration according to the embodiments can be added, deleted, or replaced with other configurations.

What is claimed is:

1. A gas detection system comprising:
an element array including a plurality of gas detection elements having different characteristic sensitivities;
a conversion unit configured to convert each of output values acquired from the gas detection elements constituting the element array into an output ratio as a ratio of the output value to a predetermined value; and
a determination unit configured to determine, by comparing a normal range generated based on a result of executing first pattern classification on a past output ratio acquired from each of the gas detection elements constituting the element array in the past with the output ratio obtained by the conversion unit executing the conversion, whether the output ratio is within the normal range.

2. The gas detection system according to claim 1, wherein
among the gas detection elements constituting the element array, the gas detection element having a largest variation range of the output ratio in a measurement period of the plurality of gas detection elements is set as a reference element, and
the conversion unit aligns the output values of the gas detection elements constituting the element array with reference to an output value of the reference element, and calculates the output ratio for each of the gas detection elements based on the aligned output values of the gas detection elements constituting the element array.

3. The gas detection system according to claim 1, wherein
the first pattern classification is cluster analysis.

4. The gas detection system according to claim 3, wherein
as a result of the cluster analysis, a plurality of clusters including the output ratio are generated,
a level is set for each cluster, and
the gas detection system further comprises a level processing unit configured to determine which cluster the current output ratio belongs to, and to execute processing corresponding to the level of the cluster to which the current output ratio belongs.

5. The gas detection system according to claim 1, further comprising
a cluster processing unit configured to execute second pattern classification when the output ratio is out of the normal range as a result of the determination executed by the determination unit.

6. The gas detection system according to claim 5, further comprising
a notification unit configured to issue an alarm when the output ratio is classified into another category that does not belong to any of patterns as a result of the second pattern classification.

7. The gas detection system according to claim 1, further comprising
a notification unit configured to issue an alarm when an odor is detected as an unusual odor as a result of comparison between a current output ratio and unusual odor data, the unusual odor data storing data related to an output ratio determined to be information on an unusual odor in the past.

8. A gas detection method comprising:
a conversion step of converting, by a gas detection system that includes an element array including a plurality of gas detection elements having different characteristic sensitivities, each of output values acquired from the gas detection elements constituting the element array into an output ratio as a ratio of an output value to a predetermined value; and
a determination step of determining, by the gas detection system comparing a normal range generated based on a result of executing first pattern classification on a past output ratio acquired from each of the gas detection elements constituting the element array in the past with the output ratio obtained by executing the conversion in the conversion step, whether the output ratio is within the normal range.
